(19) United States
(12) Patent Application Publication
(10) Pub. No.: US 2022/0054115 A1
(43) Pub. Date: Feb. 24, 2022

(54) Systems and Methods for Cell Collection and Protection of Harvested Cells

(71) Applicant: PAVmed Inc., New York, NY (US)

(72) Inventors: Lishan Aklog, Purchase, NY (US); Brian J. deGuzman, Paradise Valley, AZ (US); Richard Yazbeck, Norwell, MA (US); Michael Boutillette, San Francisco, CA (US); Jessie Gifford, New York, NY (US)

(21) Appl. No.: 17/404,746

(22) Filed: Aug. 17, 2021

Related U.S. Application Data
(60) Provisional application No. 63/067,620, filed on Aug. 19, 2020.

(51) Int. Cl. A61B 10/04

(52) U.S. Cl. CPC A61B 10/04; A61B 2010/0208

(57) ABSTRACT

Methods and systems for cell collection. Methods and systems include a collection mechanism being transformable between a collection state having an expanded diameter and a retrieval state where the diameter is less than that of the collection state and a conveyance mechanism having a distal end to which the collection mechanism is attached and designed to place the collection mechanism at a site of interest for cell collection. The Methods and systems also include an activation mechanism to transform the collection mechanism between the collection state and the retrieval state.

### RELATED U.S. APPLICATIONS

This application claims priority to U.S. Provisional Patent Application Ser. No. 63/067,620 filed on Aug. 19, 2020, which application is hereby incorporated herein in its entirety by reference.

### FIELD OF THE INVENTION

The present disclosure relates to systems and methods suitable for collection of materials and protection of the collected materials. In particular, the present disclosure relates to systems and methods that can be used to collect cells then protect the collected cells during removal from within a body.

### BACKGROUND

Generally, it is difficult to obtain desired materials and cells from patients in a simplified and pain free manner.

### SUMMARY

There is a need for improvements for cell collection and the protection of collected cell. The present disclosure is directed toward further solutions to address this need, in addition to having other desirable characteristics.

In accordance with example embodiments of the present invention a system for cell collection is provided. The system includes a collection mechanism being transformable between a collection state having an expanded diameter and a retrieval state where the diameter is less than that of the collection state and a conveyance mechanism having a distal end to which the collection mechanism is attached and designed to place the collection mechanism at a site of interest for cell collection. The system also includes an activation mechanism to transform the collection mechanism between the collection state and the retrieval state.

In accordance with aspects of the present invention, the system further includes an activation mechanism to control transformation of the cell collection mechanism between the at least two of the navigation state, the collection state, and the retrieval state. The cell collection mechanism can include an expandable material having a cell collection surface and a plurality of rings spaced along the expandable material and situated circumferentially around the expandable material, where the expandable material is designed to expand from a first circumference less than a circumference of the plurality of rings to a second circumference greater than the circumference of the plurality of rings.

In accordance with aspects of the present invention, the cell collection mechanism is a tubular construction with a plurality of axial cuts to allow the cell collection mechanism to bow. The tubular construction can be designed to bow upon application of a lateral force applied toward a central axis of the tubular construction. The tubular construction can be designed to bow upon application of an internal force applied axially outward from within the tubular construction. The internal force can be applied by an expandable material situated within the tubular construction. The tubular construction can be made from a metallic material, including nitinol. In one embodiment, the tubular construction can be elastic nitinol cut struts that bow and expand in the collection state. The tubular material is constructed with a plurality of creases and bends that are expandable into a shape for cell collection in the collection state and retracted for the retrieval state. The cell collection mechanism is a rigid tubular construction with a plurality slots to allow an internal expandable material to push through and expand out when in the collection state. The internal expandable material can retract into the plurality slots and within the rigid tubular construction when in the retrieval state.

In accordance with aspects of the present invention, the cell collection mechanism is a tubular material constructed from a plurality of tines that fan out circumferentially when in the collection state. An expandable material can control the fan out of the plurality of tines. A sheath can be situated to slide over the plurality of tines to control the fan out of the plurality of tines. Distal ends of the plurality of tines can retract to a smaller diameter than a remainder of the plurality of tines in the retrieval state. The cell collection mechanism can include a conveyance mechanism, such as a catheter shaft, a mandrel rod, and a plurality of spring steel arms for sampling.

In accordance with example embodiments of the present invention, a cell collection device is provided. The device includes a cell collection mechanism having a proximal end and a distal end, the cell collection mechanism being transformable between at least one of a navigation state, a collection state, and a retrieval state and a conveyance mechanism having a proximal end and a distal end, the distal end of the conveyance mechanism coupled to the proximal end of the cell collection mechanism.

In accordance with example embodiments of the present invention, a method for cell collection is provided. The method includes inserting a cell collection mechanism within a body in a navigation state, the cell collection mechanism being transformable between at least one of the navigation state, a collection state, and a retrieval state, navigating the cell collection mechanism to a target site for cell collection, and activating am activation mechanism to transform of the cell collection mechanism between navigation state to the collection state. The method also includes moving the cell collection mechanism at the cell collection site to gather cells on at least one surface of the cell collection mechanism, activating the activation mechanism to transform of the cell collection mechanism between the collection state and the retrieval state to protect the collected cells, and removing the cell collection mechanism.

In accordance with aspects of the present invention, while the cell collection mechanism is in the retrieval state, the cell collection mechanism provides protection of the collected cells from dilution and contamination during retrieval.

### BRIEF DESCRIPTION OF THE FIGURES

These and other characteristics of the present disclosure will be more fully understood by reference to the following detailed description in conjunction with the attached drawings, in which:

FIGS. 1A and 1B are illustrative views of a collection system for use in accordance with the present disclosure;

FIGS. 2A, 2B, and 2C are side views of a collection mechanism for use in accordance with the present disclosure;

FIGS. 3A is a side view of a collection mechanism for use in accordance with the present disclosure;

FIGS. 3B and 3C are cross sectional end views of a collection mechanism for use in accordance with the present disclosure;

FIGS. 4A is a side view of a collection mechanism for use in accordance with the present disclosure;

FIGS. 4B, 4C, and 4D are cross sectional end views of a collection mechanism for use in accordance with the present disclosure;

FIGS. 5A, 5B, and 5C are side views of various collection mechanisms for use in accordance with the present disclosure;

FIG. 6 is a side view of a collection mechanism for use in accordance with the present disclosure;

FIGS. 7A, 7B, 7C and 7D are side views of a collection mechanism for use in accordance with the present disclosure;

FIGS. 8A, 8B, and 8C illustrate a collection mechanism in accordance with another embodiment of the present invention;

FIGS. 9A and 9B illustrate a system, in accordance with one embodiment of the present invention, having an anchoring device adjacent to a collection mechanism to minimize movement of the collection mechanism.

### DETAILED DESCRIPTION

An illustrative embodiment of the present disclosure relates to systems and methods suitable for collection of materials and protection of the collected materials. In particular, the present disclosure relates to systems and methods that can be used to collect then protect collected cells from within a body. The system of the present disclosure can be used to insert a collection mechanism to a target location within a body to be activated for cell collection. The collection mechanism can be transformed between various states for different functions. For example, the collection mechanism can be transformed into any combination of a navigation state, a collection state, and a retrieval state. The navigation state can include transforming the collection mechanism into a compact shape that is suitable for navigation within a body lumen without getting caught, damaging, or otherwise interfering with the function of the body lumen. The collection state can include modifying the size and/shape of the collection mechanism to enable at least one surface of the collection mechanism to contact and sample materials from the body lumen. This state can include expanding a size and shape of a sampling surface of the collection mechanism. Once collection is complete, the collection mechanism can be transformed to a retrieval state to protect any collected materials while the system is removed from the body lumen. Similar to the navigation state, the retrieval state transforming the collection mechanism into a compact shape that is suitable for navigation within a body lumen without getting caught, damaging, or otherwise interfering with the function of the body lumen during removal of the system. In some instances, the navigation state and the retrieval state for the collection mechanism can be substantially the same in size and shape. In other instances, retrieval state can be transformed further than the navigation state.

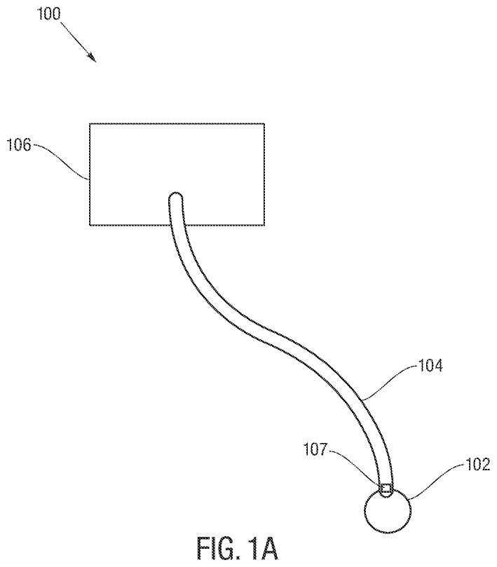

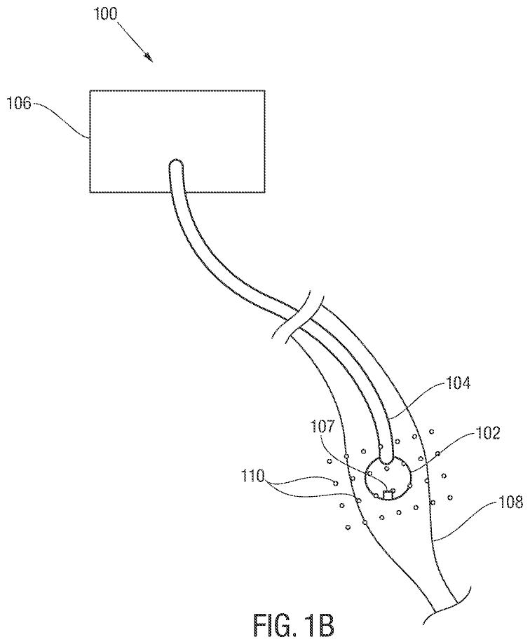

FIGS. 1A through 9B, wherein like parts are designated by like reference numerals throughout, illustrate an example embodiment or embodiments of improved operation for cell collection and protection of collected cell, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

Referring to FIGS. 1A and 1B, in some embodiments, a system 100 can be provided for inserting a collection mechanism 102 into a body 108, controlling the state of the collection mechanism 102, and removing the collection mechanism 102 from the body 108 once collection is complete. To facilitate insertion and navigation of the collection mechanism 102 into the body 108 and to a targeted site for cell collection, the system 100 can include a conveyance mechanism 104, such as an elongated catheter or catheter shaft, coupled to the collection mechanism 102. In some embodiments, the system 100 can also include an activation mechanism 106 for assisting in the navigation of the collection mechanism 102, activation of one or more features of the collection mechanism 102, and/or deactivation of the collection mechanism 102. The system 100 can be used with any combination of systems to assist in the navigation and use of the system 100 along with the localization and positioning of collection mechanism 102 at the targeted site. For example, system 100 can be aided by use of an ultrasound, endoscopy, fluoroscopy, etc. In addition to or alternatively, to aid in the navigation, localization and/or positioning of the collection mechanism 102 as the system 100 is being advanced to a targeted site, system 100 may be provided with an imaging device 107, such as a camera, proximate to collection mechanism 102. In one embodiment, imaging device 107 can be situated at a junction between the collection mechanism 102 and the conveyance mechanism 104. Placement of imaging device 107 thereat can enhance the ability of an operator to localize and position the collection mechanism 102 at the targeted site. Of course, the placement of the imaging device 107 can be anywhere along system 100, so long as imaging device 107 can be used to aid in the localization and positioning of the collection mechanism at the targeted site.

In some embodiments, the collection mechanism 102 can be a structure that can transform between different states to perform different functions. For example, the collection mechanism 102 can be designed to be transformable between at least two of the following states, a navigation state, a collection state, and a retrieval state. In the navigation state, the collection mechanism 102 can be in a size and shape to enable safe traversal within a body, for example, a reduced size for navigation within a body lumen, to be swallowed by a patient, or be intubated in a patient. In the collection state, the collection mechanism 102 can be in a size and shape to enable contact with at least one surface within a lumen of body 108, for example, an increased size for contact with one or more surfaces of the body 108 lumen. In some embodiments, in the collection state, the collection mechanism 102 can be expanded to have a diameter that is larger than a diameter of the retrieval state, where the diameter is less than that of the collection state. In the retrieval state, the collection mechanism 102 can be in a size and shape to enable both protection of collected matter and removal of the collection mechanism 102 from the body 108, for example, a reduced size for navigation out of the body 108 lumen. Each of the different states can have different combinations of sizes and shapes for the collection mechanism 102.

For example, the collection mechanism 102 can include any combination of designs capable of collecting cells 110 from the body 108 and then being transformed into a protective state for removal from the body without losing the collected cells 110. The For example, the collection mechanism 102 have a modifiable shape or surface that can be transforming between a navigation state for traversing within the body 102, to a cell collection state for collecting cells 110, and to a retrieval state for protecting collected cells 110. In addition to the collection mechanism 102, the system 100 can include any combination of structures designed to enable the insertion of, control of, and removal of the collection mechanism 102.

Continuing with FIGS. 1A and 1B in some embodiments, the conveyance mechanism 104 can be designed to assist in the transformation of the collection mechanism 102. The conveyance mechanism 104 can include a number of different mechanisms to cause the collection mechanism 102 to change between states and can vary based on the specific design of the collection mechanism 102. In some embodiments, the conveyance mechanism 104 can include a lumen to transfer or remove fluid to and from the collection mechanism 102 to cause an actuation, inflation, transition of states of the collection mechanism 102. For example, the conveyance mechanism 104 can receive fluid (e.g., liquid, gas, etc.) from the activation mechanism 106 and transfer the fluid into or out of the collection mechanism 102 for activation or deactivation. In some embodiments, the conveyance mechanism 104 can be or otherwise include a mechanical mechanism for activation or deactivation of the collection mechanism 102. For example, the conveyance mechanism 104 can include a tether, sheath, etc. that movement thereof mechanically modifies the state of the collection mechanism 102. The conveyance mechanism 104, in another embodiment, can include or otherwise be a wire or any mechanism which can deliver heat energy to the collection mechanism 102 to expand the collection mechanism 102 from a collapsed state. In addition to assisting in the transformation of the collection mechanism 102, the conveyance mechanism 104 can include any combination of structures that can transport the collection mechanism 102 into a body.

In some embodiments, the conveyance mechanism 104 can also act as or otherwise be coupled to the activation mechanism 106 for activating or deactivating one or more features of the collection mechanism 102. The reservoir of the activation mechanism 106 can be any combination of passive or active sources for providing activation means through the conveyance mechanism 104. For example, the activation mechanism 106 can include a reservoir containing fluids (e.g., liquid, gas, etc.) to be pumped through the conveyance mechanism 104 into or out of the collection mechanism 102. The activation mechanism 106 can include a pump of otherwise be connected to a separate pumping system.

The activation mechanism 106 can include any combination of components or controls for initiating and executing the transformation of the collection mechanism 102, via the conveyance mechanism 104. In one embodiment, the activation mechanism 106 can include any handheld mechanical controls or components that can be manually controlled or manipulated to modify the state of the collection mechanism 102. In another embodiment, the activation mechanism 106 can include any combination of activation buttons, switches, inputs, etc. to initiate a pump for pumping fluid through the conveyance mechanism 104 to transform the collection mechanism 102 between states. Moreover, the activation mechanism 106 can include any combination of manual or automated mechanisms. to modify that state of the collection mechanism 102. Alternatively, or in addition to, the activation mechanism 106 can include a sheath that can manually slide up and down the conveyance mechanism 104 and/or collection mechanism 102 to control transformation of the collection mechanism 102. The activation mechanism 106 can also include a source to activate the heat energy to be delivered to the collection mechanism 102. The activation mechanism 106 can be used to control transformation of the collection mechanism 102 between the at least two of the following states, specifically the navigation state, the collection state, and the retrieval state.

The collection mechanism 102, the conveyance mechanism 104, and the activation mechanism 106 can be separate or integrated components coupled together to form the system 100. In some embodiments, the collection mechanism 102 and the conveyance mechanism 104 can have a proximal end and a distal end with the distal end of the conveyance mechanism 104 being designed to couple to the proximal end of the cell collection mechanism 102. The components can be coupled together using any combination of methods known in the art, for example, welding, adhesive, mechanical coupling, etc. The collection mechanism 102 and the conveyance mechanism 104 can be removably or fixedly coupled together. In some embodiments, the conveyance mechanism 104 can be designed to place the collection mechanism 102 at a site of interest for cell collection. For example, the conveyance mechanism 104 can be designed to have sufficient flexibility to be able to navigate a curved channel while also having sufficient rigidity to be advanced through the channel (e.g., by application of a pushing force). The collection mechanism 102 and the conveyance mechanism 104 can be constructed from any combination of materials that are safe for insertion and navigation in the body 108. For example, the collection mechanism 102 and conveyance mechanism 104 can be constructed from a biomedically safe material.

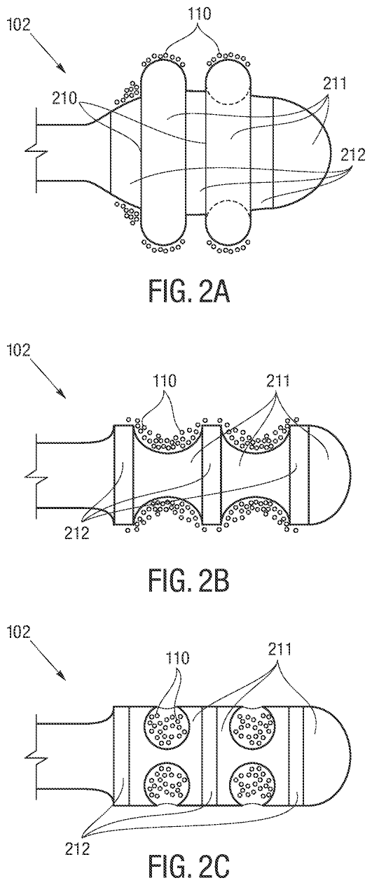

Referring to FIGS. 2A-2C, in some embodiments, the collection mechanism 102 can be constructed from an expandable material 210 and a plurality of rings 212 or ridges constructed from a substantially rigid material. The expandable material 210 can include any combination of materials that can expand upon the application of force. For example, the expandable material 210 can be a silicon or other elastic type material. In some embodiments, the expandable material 210 can be in fluid communication with the conveyance mechanism 104 such that the conveyance mechanism 104 can be used to pressurize or depressurize the expandable material 210 to cause the expandable material 210 to expand or retract, respectively. For example, the conveyance mechanism 104 can receive and transfer fluid into an interior cavity of the expandable material 210 in response to an activation of the activation mechanism 106 to pressurize and expand or depressurize and retract the expandable material 210. Alternatively, or in addition to, the expandable material 210 from which collection mechanism 102 is made can be a silicon or elastic material that expand upon exposure to heat energy or be any material with similar properties.

In some embodiments, in at least one state, the expandable material 210 can be surrounded by or otherwise coupled to the plurality of rings 212. In other words, the plurality of rings 212 can be spaced along the expandable material 210 and situated circumferentially around the expandable material 210. For example, the expandable material 210 can be a single inflatable component positioned within a linear circumference of the plurality of rings 212 or the collection mechanism can be a plurality of expandable components individually attached between each of the plurality of rings 212. The plurality of rings 212 can be spaced to provide sufficient room for the expandable material 210 to expand through and outward beyond the dimensions of the plurality of rings 212, in response to pressurization of the collection mechanism 102. Similarly, the plurality of rings 212 can be sufficiently rigid to maintain their shape as the expandable material 210 expands therethrough. In some embodiments, the expandable material 210 can be designed to expand from a first circumference that less than a circumference of the plurality of rings 212 to a second circumference that is greater than the circumference of the plurality of rings 212.

Continuing with FIGS. 2A-2C, in some embodiments, the collection mechanism 102 can have a collection surface 211 about a periphery of the collection mechanism 102, for example, designed to collect cells 110. The collection surface 211 can include the expandable material 210 having any combination of textures, surface materials, shapes, etc. designed to capture a desired material(s). The expandable material 210 can be designed to both remove and adhere material to its surface and maintain the material on its surface for retrieval. Examples of such materials can include, but not limited to, shape memory foam, open cell foam, knitted materials, various fabric materials, hook and loop systems, gauze or any other roughened or textured surface materials that can facilitate capturing, adherence, and retention of the desired materials for retrieval purposes. In some embodiments, the collection mechanism 102 can include a coating to assist in navigation within a body and/or collection of material. For example, the plurality of rings 212 can have a lubricated coating for navigation and movement of the expandable material 210 and the expandable material 210 can include a coating that attracts/adheres to targeted cells 110.

The combination of the conveyance mechanism 104, expandable material 210 and the plurality of rings 212 can be used to transition through the different states of the system 100 to carry out the collection process of the present disclosure. Referring to FIG. 2A, once the system 100 is positioned at a desired site for collection (e.g., within body 108) the collection mechanism 102 can be transitioned into a collection state. To transform the collection mechanism 102 into the collection state, in some embodiments, pressure can be applied to the expandable material 210, via the conveyance mechanism 104, to expand the expandable material 210 through gaps between the plurality of rings 212. The expandable material 210 can continue to expand outside the area defined by the plurality of rings 212 to a sized with a larger circumference than the plurality of rings 212. With the expandable material 210 in an expanded shape, as depicted in FIG. 2A, the surface of the expandable material 210 can be placed into contact with a side of the body to collect material thereon. For example, the collection mechanism 102 can be inserted into a body 108 lumen and pressurized to expand and contact a sidewall of the body 108 to collect cells 110 on a surface of the expandable material 210.

Referring to FIGS. 2B and 2C, in some embodiments, once collection has taken place, the expandable member 210 can be transitioned to a retrieval state. To transform the collection mechanism 102 into the retrieval state, in some embodiments, negative pressure can be applied to the expandable material 210 (or removal of an application of a positive pressure), via the conveyance mechanism 104, to contract the expandable material 210 through gaps between the plurality of rings 212. The expandable material 210 can continue to contract into the area defined by the plurality of rings 212 to a sized with a smaller circumference than that of the plurality of rings 212. With the expandable material 210 in a contracted or relaxed shape, as depicted in FIG. 2B, the collected material on the surface of the expandable material 210 can be protected during removal of the collection mechanism 102. For example, cells 110 adhered to the surface of the expandable material 210 can be pulled with the expandable material into recess created between the plurality of rings 212.

Referring to FIG. 2C, in some embodiments, a further negative pressure can be applied to the expandable material 210, via the conveyance mechanism 104, to further contract the expandable material 210 and/or compress an overall length of the collection mechanism 102. This negative application of pressure can cause the collection mechanism 102, and particularly the expandable material 210, to contract in two directions. For example, a sufficient negative pressure can be applied to the interior of the expandable material 210 to cause the expandable material to constrict radially such that it also pulls the plurality of rings 212 linearly toward one another to reduce an amount of space between the plurality of rings 212 and thus reduce an area exposed to the body 108. As shown in FIG. 2C, by further contracting the expandable material 210, collected sample materials can be further protected by creating substantially encapsulated pockets. For example, cells 110 adhered to the surface of the expandable material 210 can be pulled with the expandable material into pockets created between the plurality of rings 212. The pockets can be completely encapsulated or substantially encapsulated to limit potential loss of the collected material on the expandable material 110. Similar functionality can be provided when applying positive pressure to cause the collection mechanism 102 to expand in two directions.

The application of positive and negative pressure can be applied using any combination of systems or methods known in the art. For example, any combination of pumps, hydraulics, pistons, etc. can be activated through any combination automatic or manual systems can be used. For example, a syringe can be used to add fluid into the collection mechanism 102 (via conveyance mechanism 104) for a positive pressure and to remove fluid from the collection mechanism 102 for a negative pressure.

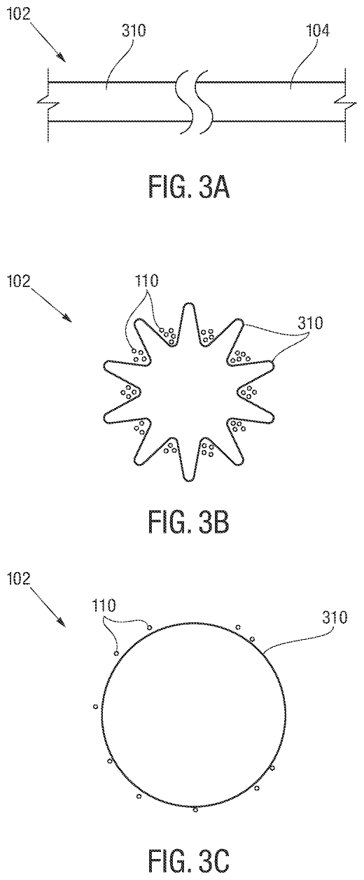

Referring to FIGS. 3A-3C, in some embodiments, the collection mechanism 102 can be an expandable member 310 with shape memory that is able to transition between different shapes, for the various states, in response to changes in an applied pressure or force. The shape memory expandable material 310 can include any combination or symmetric or asymmetric shapes that can be used for a navigation state, a collection state, and a retrieval state. The expandable material 310 can include any combination of materials that can expand upon the application of force. For example, the expandable material 310 can be a silicon or other elastic type material. In some embodiments, the expandable material 310 can be in fluid communication with the conveyance mechanism 104 such that the conveyance mechanism 104 can be used to pressurize or depressurize the expandable material 310 to cause the expandable material 310 to expand or retract, respectively. For example, the conveyance mechanism 104 can receive and transfer fluid into an interior cavity of the expandable material 310 in response to an activation of the activation mechanism 106 to pressurize and expand or depressurize and retract the expandable material 310. Alternatively, or in addition to, the expandable material 310 from which collection mechanism 102 is made can be a silicon or elastic material that expand upon exposure to heat energy or be any material with similar properties.

Referring to FIG. 3A, in some embodiments, the collection mechanism 102 can be a tubular shape constructed formed from an expandable material 310. The tubular shaped collection mechanism 102 can be an extension of the conveyance mechanism 104 or it can be a separate component attached to a distal end of the conveyance mechanism 104.

Referring to FIGS. 3B and 3C, the collection mechanism 102 can transition from a navigation state/retrieval state to a collection state. In some embodiments, the navigation state/retrieval state of the collection mechanism 102 can be when the expandable material 310 is in a relaxed state having a plurality of creases and bends positioned about a circumference of the collection mechanism 102, as shown in FIG. 3B. When a positive pressure is applied to the collection mechanism 102, the expandable material 310 can expand such that the creases and bends are pushed outward radially to create a sufficiently uniform surface that is suitable for collection, as shown in FIG. 3C. For collection, the expandable material 310 can be designed with any combination of textures, surface materials, shapes, etc. designed to capture a desired material(s), adhere material to its surface, and maintain the material on its surface for retrieval. Examples of such materials can include, but not limited to, shape memory foam, open cell foam, knitted materials, various fabric materials, hook and loop systems, gauze or any other roughened or textured surface materials that can facilitate capturing, adherence, and retention of the desired materials for retrieval purposes. The collection mechanism 102 can also include a coating to assist in navigation within a body and/or collection of material. In some embodiments, the surface type and coatings on the expandable material 310 can vary based on location and function. For example, the plurality of creases can include a texture and/or coating that attracts/adheres to targeted cells 110 when the expandable material 310 is in its expanded state.

In some embodiments, after the collection mechanism 102 has been used to collect material on the expandable material 310, it can be transformed into the navigation state/retrieval state for safe removal. For example, a negative pressure (or removal of a positive pressure) can be applied to the collection mechanism 102 to retract/revert the expandable material 310 back to its neutral shape, as shown in FIG. 3B. When in its neutral shape, the expandable material 310 can include a plurality of creases which can provide protection to any collected materials on the surface of the expandable material 310. The application of positive and negative pressure can be applied using any combination of systems or methods known in the art. For example, any combination of pumps, hydraulics, pistons, etc. can be activated through any combination automatic or manual systems can be used. For example, a syringe can be used to add fluid into the collection mechanism 102 (via conveyance mechanism 104) for a positive pressure and to remove fluid from the collection mechanism 102 for a negative pressure.

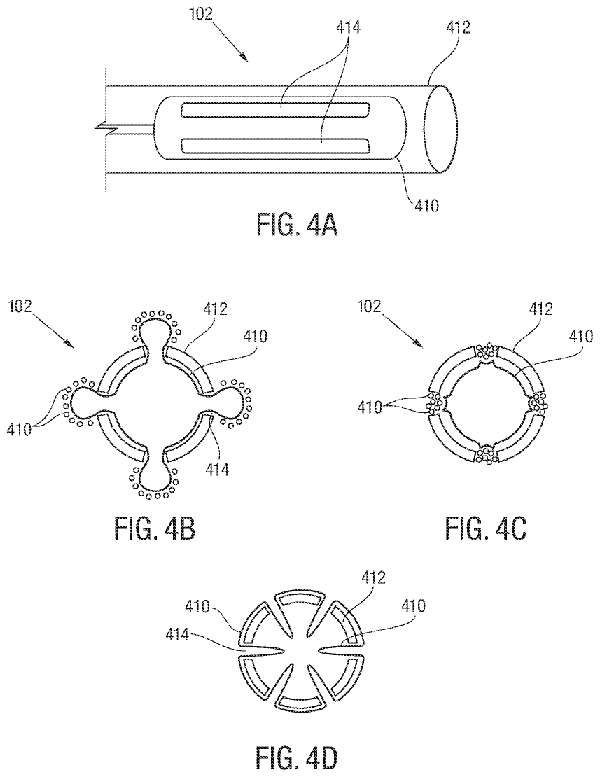

Referring now to FIGS. 4A-4D, in some embodiments, the collection mechanism 102 can include a tubular construction 412 with a plurality of slots 414 therethrough. The tubular construction 412 can be a constructed from a rigid material, such as a stainless-steel tube. In some embodiments, the tubular construction 412 can house and be situated circumferentially around an expandable material 410. In some embodiments, in at least one state, the expandable material 210 can be surrounded by or otherwise coupled to an inner surface of the tubular construction 412. The expandable material 410 can include any combination of materials that can expand upon the application of force. For example, the expandable material 410 can be a silicon or other elastic type material. Alternatively, or in addition to, the expandable material 410 from which collection mechanism 102 is made can be a silicon or elastic material that expand upon exposure to heat energy or be any material with similar properties.

In some embodiments, the expandable material 410 can be a custom shaped balloon that will expand into a particular shape. For example, as shown in FIG. 4B, the expandable material 410 can be a four-leaf clover shape when fully expanded. In some embodiments, the expandable material 410 can be in fluid communication with the conveyance mechanism 104 such that the conveyance mechanism 104 can be used to pressurize or depressurize the expandable material 410 to cause the expandable material 410 to expand or retract, respectively. For example, the conveyance mechanism 104 can receive and transfer fluid into an interior cavity of the expandable material 410 in response to an activation of the activation mechanism 106 to pressurize and expand or depressurize and retract the expandable material 410. Alternatively, when the expandable material 410 includes a heat expandable material, conveyance mechanism 104 can be used to direct or terminate heat energy to the expandable material 410 in order to bias it between an expanded and a collapsed state.

In some embodiments, the plurality slots 414 can be sized and shaped to allow an expandable material 410 to push from within the tubular construction 412, through and expand out of the tubular construction 412 when transitioned into the collection state. The plurality slots 414 can include any combination of shapes to allow the expandable material 410 to protrude therethrough. The plurality of plurality slots 414 can be spaced to provide sufficient room for the expandable material 210 to expand through and outward beyond the dimensions of the plurality slots 414, in response to pressurization. Similarly, the tubular construction 412 can be sufficiently rigid to maintain their shape as the expandable material 410 expands therethrough. In some embodiments, the expandable material 410 can be designed to expand from a first circumference that less than a circumference of the tubular construction 412 to a second circumference that is greater than the circumference of the tubular construction 412. Depending on the shape of the expandable material 410, once portions of the expandable material has expanded beyond the plurality slots 414, it can be further expanded out radially, for example, as shown in FIG. 4B. The radial expansion can provide a greater surface area for collection on the expandable material 410.

Continuing with FIGS. 4A-4C, in some embodiments, the collection mechanism 102 can have a collection surface, for example, to collect cells 110. The collection surface can include the expandable material 410 having any combination of textures, surface materials, shapes, etc. designed to capture a desired material(s). Examples of such materials can include, but not limited to, shape memory foam, open cell foam, knitted materials, various fabric materials, hook and loop systems, gauze or any other roughened or textured surface materials that can facilitate capturing, adherence, and retention of the desired materials for retrieval purposes. The expandable material 410 can be designed to both remove and adhere material to its surface and maintain the material on its surface for retrieval. In some embodiments, the collection mechanism 102 can include a coating to assist in navigation within a body and/or collection of material. For example, the tubular construction 412 can have a lubricated coating for navigation and movement of the expandable material 410 and the expandable material 410 can include a coating that attracts/adheres to targeted cells 110.

In some embodiments, the internal expandable material 410 can be designed to retract into the plurality slots 414 and within the tubular construction 412 when transitioned into the retrieval state, for example, as shown in FIG. 4C. When in the retrieval state, the collected materials can be protected during removal of the collection device 102 from the body 108.

The combination of the conveyance mechanism 104, the tubular construction 412, and the expandable material 410 can be used to transition through the different states of the system 100 to carry out the collection process of the present disclosure. Referring to FIG. 4B, once the system 100 is positioned at a desired site for collection (e.g., within body 108) the collection mechanism 102 can be transitioned into a collection state. To transform the collection mechanism 102 into the collection state, in some embodiments, pressure or heat energy can be applied to the expandable material 410, via the conveyance mechanism 104, to expand the expandable material 410 through the plurality slots 414 between the tubular construction 412. The expandable material 410 can continue to expand outside the area defined by the plurality slots 414 to a sized with a larger circumference than the tubular construction 412. With the expandable material 410 in an expanded shape, as depicted in FIG. 4B, the surface of the expandable material 410 can be placed into contact with a side of the body 108 to collect material thereon. For example, the collection mechanism 102 can be inserted into a body 108 lumen and pressurized to expand and contact a sidewall of the body 108 to collect cells 110 on a surface of the expandable material 210.

Referring to FIGS. 4C, in some embodiments, once collection has taken place, the expandable member 410 can be transitioned to a retrieval state. To transform the collection mechanism 102 into the retrieval state, in some embodiments, negative pressure can be applied to the expandable material 410 (or removal of an application of a positive pressure) or heat energy to the expandable material 410 can be terminated, via the conveyance mechanism 104, to contract the expandable material 410 through the plurality slots 414 in the tubular construction 412. The expandable material 410 can continue to contract into the interior area defined by the tubular construction 412 to a size with a smaller circumference than the tubular construction 412. With the expandable material 410 in a contracted or relaxed shape, as depicted in FIG. 4C, the collected material on the surface of the expandable material 410 can be protected during removal of the collection mechanism 102. For example, cells 110 adhered to the surface of the expandable material 410 can be pulled with the expandable material into recess created between the plurality slots 414 for protection within the tubular construction 412.

The application of positive and negative pressure can be applied using any combination of systems or methods known in the art. For example, any combination of pumps, hydraulics, pistons, etc. can be activated through any combination automatic or manual systems can be used. For example, a syringe can be used to add fluid into the collection mechanism 102 (via conveyance mechanism 104) for a positive pressure and to remove fluid from the collection mechanism 102 for a negative pressure. Similarly, the application of heat energy to collection mechanism 102 can be provided using any combination of systems or methods known in the art.

Referring to FIG. 4D, in some embodiments, the expandable material 410 can be wrapped around the tubular construction 412 and at least partially retractable within the plurality slots 414. In this configuration, in the collection state, the expandable material 410 can expand radially outward from the tubular construction 412 and out of the plurality slots 414 to create a substantially uniform collection shape, for example, similar to the shape provided in FIG. 3C. In the configuration of FIG. 4D, the expandable material 410 can take any combination of shapes when in the collection state. Regardless of shape, once materials have been collected on the surface of the elastic member, a negative pressure can be applied or heat energy can be terminated to retract the expandable material 410 substantially around the tubular construction 412 and within plurality slots 414, as discussed with respect to FIGS. 4A-4C.

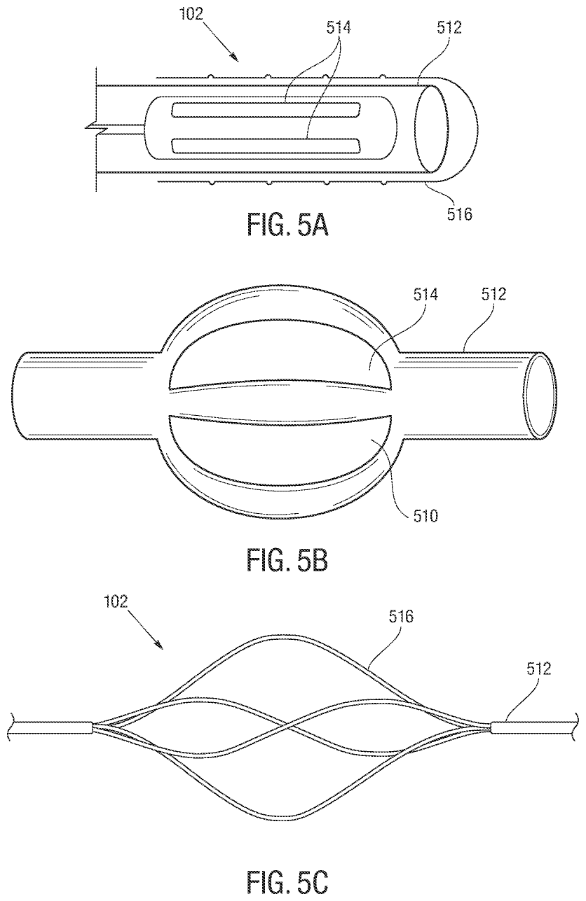

Referring to FIGS. 5A-5C, in some embodiments, the collection mechanism 102 can be a tubular construction 512 with a plurality of struts, such as axial cuts 514, situated therein. The tubular construction 512 can be constructed from any combination of materials that enables the tubular construction 512 to bend or flex in response to an application of force. For example, the tubular construction 512 can be made from a metallic material, including nitinol. In one embodiment, the tubular construction 512 can be made from elastic nitinol cut struts that bow and expand. The plurality of axial cuts 514 can be sized and shaped to allow the tubular construction 512 to bow, for example, as shown in FIG. 5B. In an alternative embodiment, tubular construction 512, instead of being provided with axial cuts 514, can be provided with helical struts 516, as shown in FIG. 5C.

In some embodiments, the tubular construction 512 can be designed to bow upon application of a force applied along a central axis of the tubular construction 512. For example, this force (i.e., axial force) can be applied to the distal end and/or the proximal end of the to push the distal and proximal ends linearly toward one another along the central axis. When the distal and proximal ends of the tubular construction 512 are pushed/pulled linearly toward one another along the central axis, the middle section of the tubular construction 512 can bow radially away from the central axis to accommodate the advancement of the ends. In such a design, it should be appreciated that reversion to the non-bow or collapsed state can be achieved by removal of the axial force or by pushing the distal and proximal ends away from one another along the central axis. It should also be appreciated that although shown with the designs provided in FIGS. 5B and 5C, the struts 514 or 516 on tubular construction 512 and their configuration can incorporate any designs so long as the struts can bow radially from the central axis.

In some embodiments, the tubular construction 512 can be constructed from a super elastic material designed to bow upon application of an internal force applied radially outward from within the tubular construction 512 and away from the axis. The internal force can be applied by an expandable material 510 situated within the tubular construction 512. The expandable material 510 can cause radial expansion from within the tubular construction 512. The diameter of the tubular construction 512 can correspond to the expansion of the expandable material 510. In such a design, it should be appreciated that reversion to the non-bow or collapsed state can be achieved by removal of the applied force.

In some embodiments, the expandable material 510 can be designed to expand and contract in response to a positive pressure and a negative pressure, respectively, being applied thereto. For example, the positive pressure and negative pressure can be applied to the expandable material 510 by a conveyance mechanism 104 in fluid communication with the expandable material 510, as discussed in greater detail herein. The application of positive and negative pressure can be applied using any combination of systems or methods known in the art. For example, any combination of pumps, hydraulics, pistons, etc. can be activated through any combination automatic or manual systems can be used. For example, a syringe can be used to add fluid into the collection mechanism 102 (via conveyance mechanism 104) for a positive pressure and to remove fluid from the collection mechanism 102 for a negative pressure.

Referring to FIG. 5A, the collection mechanism 102 can be transitioned between a navigation / retrieval state, as depicted in FIG. 5A, and a collection state, as depicted in FIG. 5B and FIG. 5C. When in the collection state, some combination of the tubular construction 512 and the expandable material 510 can have one or more collection surfaces. For example, the edges of the tubular construction 512 can be used to scrap of surface of the body 108 for collection of scrapped cells 110 on the expandable material 510. The tubular construction 512 and/or the expandable material 510 can be designed with any combination of textures, surface materials, shapes, etc. designed to capture a desired material(s), adhere material to its surface, and maintain the material on its surface for retrieval (e.g., in a protected retrieval state). Examples of such materials can include, but not limited to, shape memory foam, open cell foam, knitted materials, various fabric materials, hook and loop systems, gauze or any other roughened or textured surface materials that can facilitate capturing, adherence, and retention of the desired materials for retrieval purposes. The collection mechanism 102 can also include a coating to assist in navigation within a body and/or collection of material.

In some embodiments, the tubular construction 512 can be any shape memory materials known in the art that can default to a bowed state in its natural state, and a sleeve 516 can be placed over the expandable material 510 to compress the tubular construction 512 to a non-bowed or collapsed state. As such, at a targeted site when sleeve 516 is removed, the tubular construction 512 can transition back to its bowed or expanded state.

In another embodiment, the tubular construction 512 can be made from any shape memory materials, where in a natural state, tubular construction 512 is in a collapsed or non-bow state. In this embodiment, the tubular construction 512 can be made from materials known in the art, which upon application of heat energy thereto, can transition the tubular construction 512 from a collapsed state to an expanded state. Upon termination of the heat energy, the tubular construction 512 can revert back to its collapsed state. In a further embodiment, the tubular construction 512 can be made from shape memory material, where in its natural state, it is in a collapsed or non-bow state. In this embodiment, the tubular construction 512 can be made from materials known in the art, which upon exposure to heat in the surrounding proximity, can transition the tubular construction 512 from a collapsed state to an expanded state. To that end a sleeve may be used to cover the tubular construction 512 in its collapsed state to protect it from surrounding heat until exposure and subsequent expansion is desired.

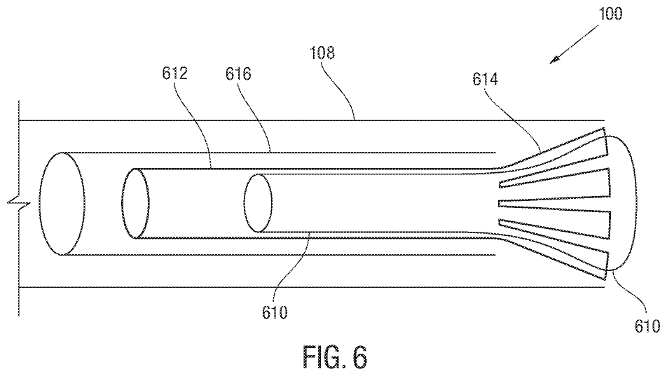

Referring to FIG. 6, in some embodiments, the collection mechanism 102 can include a tubular construction 612 that has a shape memory and a plurality of cuts that create a plurality of tines 614. For example, the tubular construction 612 can be made from a metallic material, including nitinol. In one embodiment, the tubular construction 612 can be made from elastic nitinol that has a preformed shape. In some embodiments, the plurality of tines 614 of the tubular construction 612 can be designed to naturally fan outward, as shown in FIG. 6. The tubular construction 612 can be constructed from any combination of materials that enables the plurality of tines 614 to bend or flex in response to an application of force. The plurality of tines 614 can be designed to fan out from a central axis naturally and/or in response to an application of, for example, an internal or external force, or to heat energy.

In some embodiments, the collection mechanism 102 can have a protective sheath 616 situated over the tubular construction 612 and sized and shaped to slide over the tubular construction 612 and plurality of tines 614. The sheath 616 can be constructed from any combination of materials able to control the shape of the plurality of tines 614 such that sliding the sheath 616 over the plurality of tines 614 to substantially conform the tines to the diameter of the sheath 616. Similarly, removal of the sheath 616 can cause the plurality of tines 614 to splay outward away from the main trunk of the tubular construction 612, for example, a remembered shape. In some embodiments, the distal ends of the plurality of tines 614 can be designed with a tapered shape to retract to a smaller diameter than the rest of the plurality of tines 614 to provide protection during the retrieval state.

In some embodiments, an expandable material 610 can be positioned within the tubular construction 612. The expandable material 610 can be designed to push out and control the shape of the plurality of tines 614 upon expansion. In some embodiments, the expandable material 610 can be tethered to the plurality of tines 614 to pull the plurality of tines 614 inward upon contraction of the expandable material 610. In some embodiments, the expandable material 610 can be designed to expand and contract in response to a positive pressure and a negative pressure, respectively, being applied thereto. For example, the positive pressure and negative pressure can be applied to the expandable material 610 by a conveyance mechanism 104 in fluid communication with the expandable material 610, as discussed in greater detail herein. The application of positive and negative pressure can be applied using any combination of systems or methods known in the art. For example, any combination of pumps, hydraulics, pistons, etc. can be activated through any combination automatic or manual systems can be used. For example, a syringe can be used to add fluid into the collection mechanism 102 (via conveyance mechanism 104) for a positive pressure and to remove fluid from the collection mechanism 102 for a negative pressure.

Referring to FIG. 6, the collection mechanism 102 can be transitioned between a navigation/retrieval state and a collection state, as depicted in FIG. 6. Transitioning from a navigation state to a collection state can be achieved by any combination of sliding the sheath 616 away from the plurality of tines 614 to allow the plurality of tines 614 to fan out or pressurizing the expandable material 610 to push the plurality of tines 614 outward. When in the collection state, some combination of the plurality of tines 614 and the expandable material 610 can have one or more collection surfaces. The tubular construction 612, the plurality of tines 614, and/or the expandable material 610 can be designed with any combination of textures, surface materials, shapes, etc. designed to capture a desired material(s), adhere material to its surface, and maintain the material on its surface for retrieval. The collection mechanism 102 can also include a coating to assist in navigation within a body and/or collection of material. For example, the non-tines portion of the tubular member 612 can include a lubricating coating. Both the plurality of tines 614 and the expandable material 610 can be designed to work in combination for cell collection. For example, the edges of the plurality of tines 614 can be used to scrap of surface of the body 108 for collection of scrapped cells 110 on the expandable material 610.

Continuing with FIG. 6, the collection mechanism 102 can be transitioned from the collection state back into the navigation/retrieval state. Transitioning back to the navigation state can be achieved by any combination of sliding the sheath 616 toward and over the plurality of tines 614 to allow the plurality of tines 614 to compress back to a smaller circumference or applying a negative pressurize the expandable material 610 to pull or allow the plurality of tines 614 to return inward. In some embodiments, the distal ends of the plurality of tines 614 can retract to a smaller diameter than a remainder of the plurality of tines 514 and the sheath 516 when in the retrieval state. When the distal ends of the plurality of tines 514 have a smaller circumference than the rest of the tines and the sheath 516, the compression of the plurality of tines 514 can be performed without losing collected material thereon (e.g., being scrapped off by the sheath 516). In some embodiments, the sheath 516 can be replaced by a lumen within an endoscope.

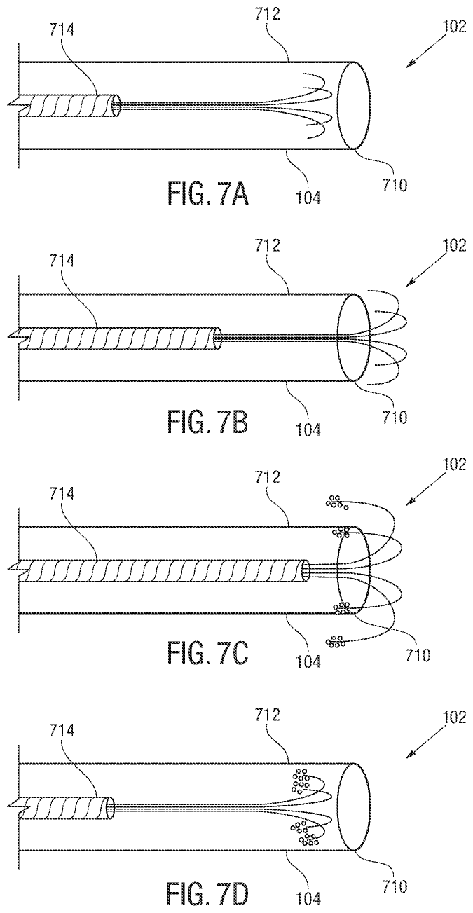

Referring to FIGS. 7A-7C, in some embodiments, the collection mechanism 102 can be positioned within distal end 712 of conveyance mechanism 104. In one embodiment, collection mechanism 102 includes a mandrel rod 714, and a plurality of spring steel arms 710 for sampling or collection of cells. The mandrel rod 714 and the plurality of spring steel arms 710 can be positioned within the distal end 712 and can be designed to move within the cavity of the conveyance mechanism 104. The plurality of spring steel arms 710 can be formed with a cell collection surface thereon. In some embodiments, the proximal ends of each of the plurality of spring steel arms 710 can be coupled to the distal end of the mandrel rod 714. The mandrel rod 714 can be used to advance the plurality of spring steel arms 710 out of the distal end 712. As the plurality of spring steel arms 710 extend out of the distal end 712, the arms 710 can fan out, for example, into a mushroom type shape, as shown in FIG. 7B. With the plurality of spring steel arms 710 spread outside the distal end 712, the plurality of spring steel arms 710 can contact its outer surfaces at the targeted site to collect materials thereon, as illustrated in FIG. 7C. Similarly, for protection and safe collection of the collected materials, the plurality of spring steel arms 710 can be retracted back into the distal end 712 (e.g., in a protected retrieval state) by pulling the mandrel rod 714 back toward the proximal end of the distal end 712, as shown in FIG. 7D.

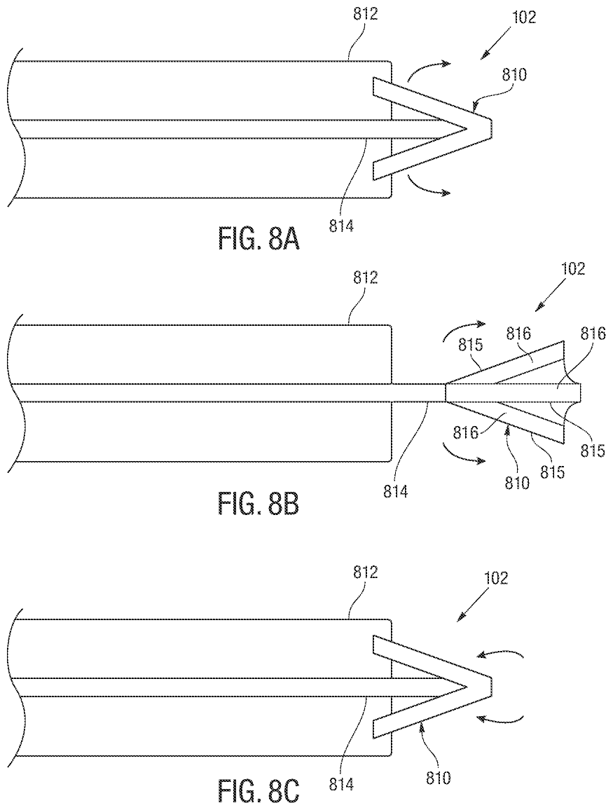

Referring now to FIG. 8A-C, there is illustrated another embodiment of a system 100 of collecting cells. System 100, in one embodiment, can be designed so that the collection mechanism 102 can be positioned within distal end 812 of conveyance mechanism 104. In one embodiment, collection mechanism 102 includes a mandrel rod 814, and an umbrella shaped mechanism 810 for sampling or collection of cells. The mandrel rod 814 and the umbrella shaped mechanism 810, as shown in FIG. 8A, can be positioned within the distal end 812 in an inverted and collapsed state, and can be designed to move and translate within the cavity of the conveyance mechanism 104. The umbrella shaped mechanism 810, as illustrated in FIG. 8B, includes, a plurality of arms 815, each having a cell collection surface 816 which can be exposed when the umbrella shaped mechanism 810 is advanced beyond distal end 812 of conveyance mechanism 104 to transition to an everted and expanded state. As illustrated, the plurality of arms 815 can be coupled at their proximal ends to a distal end of the mandrel rod 814. The mandrel rod 814 can be used, in an embodiment, to advance the umbrella shaped mechanism 810 out of the distal end 812, as shown in FIG. 8B. In the everted and expanded state beyond the distal end 812 of conveyance mechanism 104, the arms 815 can be maneuver so that their outer surfaces 816 can contact the targeted site to collect materials, for instance cells, thereon. Once the materials have been collected, for protection of the collected materials, the mandrel rod 814 can be retracted to pull the umbrella shaped mechanism 810 and its arms 815 back into the distal end 812 in an inverted and collapsed state, as shown in FIG. 8C.

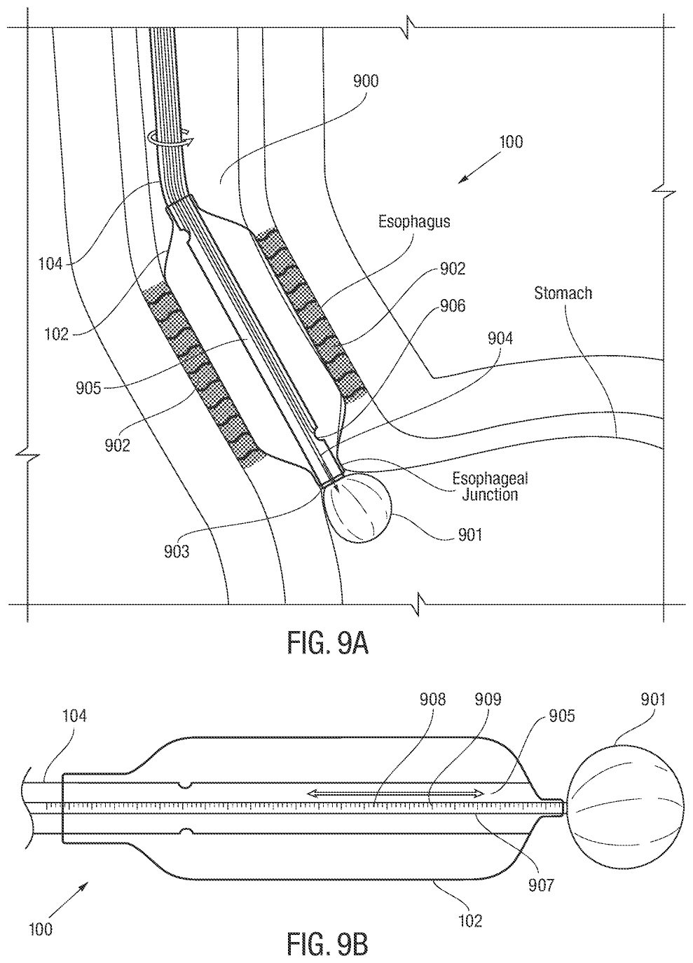

Referring now to FIGS. 9A-9B, system 100 may further include an anchoring device, such as balloon 901, configured to help anchor system 100 stay in place within body tract 900, for example the esophagus. This is because there can be a number of forces that can cause collection mechanism 102 to move or migrate away from a targeted site 902 during treatment. For example, muscle contractions such as those associated with swallowing or gagging may push or pull on collection mechanism 102 and thereby move collection mechanism up or down body tract 900. Likewise, an operator of system 100 may inadvertently push or pull on conveyance mechanism 104 during treatment, causing collection mechanism 102 to move away from the target site. This can be particularly problematic in instances in which the operator needs to collection mechanism 102 to engage the targeted site. Still further, collection mechanism 102 can often be advanced to the targeted site in a deflated state and subsequently inflated during treatment—as the collection mechanism 102 expands, it may migrate away from the targeted site, especially if that portion of tract 900 has a non-uniform diameter (e.g., narrows or widens).

Balloon 901, in one embodiment, may be axially spaced from collection mechanism 102 such that balloon 901 is positioned distal to collection mechanism 102, as shown in FIG. 9A. Balloon 901, by design, may be configured to help anchor collection mechanism 102 remain in place within body tract 900 by blocking retreat of conveyance mechanism 104 and collection mechanism 102. In such a configuration, balloon 901 may be configured to expand to dimensions relatively greater than a diameter of an opening 903 of body tract 900 such that, when positioned beyond opening 903 and expanded, balloon 901 can be prevented from being withdrawn through opening 903. Since balloon 901 and collection mechanism 102 are coupled to conveyance mechanism 104, the position of the collection mechanism 102, for instance, at the targeted site 902, can therefore be ensured within body tract 900.

By way of example, as shown in 9A, balloon 901 may be positioned beyond opening 903 at the gastroesophageal junction where the esophagus joins the stomach. Balloon 901 may be positioned in the stomach beyond the gastroesophageal junction and expanded within the stomach to dimensions greater than the diameter of the opening 902. As expanded, balloon 901 cannot fit back through the opening 902 at the gastroesophageal junction and thus balloon 901 can act to prevent conveyance mechanism 104 and collection mechanism 102 from being withdrawn up the esophagus and away from targeted site 903.

To expand balloon 901, conveyance mechanism 104, in one embodiment, may be provided with a lumen 904 in fluid communication with balloon 901 to permit fluid, e.g., gas, liquid etc., to be introduced into balloon 901 and expand balloon 901. It should be appreciated that should collection mechanism 102 be an inflatable body, lumen 904 can be provided along conveyance mechanism 104 as a separate lumen from lumen 905 which is used to introduce fluid into the collection mechanism 102 via at least one side opening 906.

Still referring to FIG. 9A, the axial distance between collection mechanism 102 and balloon 901 may be provided such that, depending on the procedure, collection mechanism 102 can be positioned adjacent targeted site 902 when balloon 901 is positioned beyond opening 903. For example, when treating the lower esophagus, the axial distance between collection mechanism 102 and balloon 901 may be relatively small since the lower esophagus is closer to opening 903 at the gastroesophageal junction and beyond which balloon 901 is to be positioned for anchoring. Conversely, when treating the upper esophagus, the axial distance between collection mechanism 102 and balloon 901 may be relatively large since the upper esophagus is farther away from opening 903 at the gastroesophageal junction beyond which balloon 901 is to be positioned for anchoring. To that end, assuming an average length of the esophagus of a patient is about 25 centimeters long, in accordance with various embodiments of the present invention, the axial distance between collection mechanism 102 and balloon 901 may range from about 0-5 centimeters when treating the lower esophagus, may range from about 5-15 centimeters when treating the mid-esophagus, and may range from about 15-25 centimeters when treating the upper esophagus. Variation in the axial distance between the collection mechanism 102 and balloon 901, of course, is contemplated within the present disclosure and one of ordinary skill in the art will recognize an appropriate axial distance for a particular application in view of the teachings of the present disclosure and general knowledge regarding human anatomy.

Referring now to FIG. 9B, in one embodiment, to facilitate varying the axial distance between collection mechanism 102 and balloon 901 in system 100, balloon 901 may be coupled to an elongated member 907 slidably situated within lumen 905 of conveyance mechanism 104 to which collection mechanism 102 is coupled. Elongated member 907, in an embodiment, can be provided with a pathway 908 along which fluid can be introduced into balloon 901 to expand balloon 901. In this embodiment, since collection mechanism 102 is coupled to conveyance mechanism 104 and balloon 901 coupled to elongated member 907 that is slidably situated within lumen 905 of conveyance mechanism 104, once balloon 901 has been expand and secured against opening 903 at the gastroesophageal junction, the axial distance between collection mechanism 102 and balloon 901 can be adjusted, prior to expand the collection mechanism 102 within the body tract, such as the esophagus, by moving the collection mechanism 102 away or toward balloon 901. It should be appreciated that should the collection mechanism 102 be an inflatable body, such as that in shown FIG. 9B, the inflatable body can be tightly sealed against conveyance mechanism 104 to ensure any leakage of fluid from within the inflatable body. Elongate member 907 may serve any one or combination of purposes including, for example, adding longitudinal/axial rigidity to collection mechanism 102, providing a conduit access to the distal portion of system 100, and/or providing a conduit for inflating and deflating a distal anchor balloon.

To the extent desired, elongated member 907 can be provided, in accordance with an embodiment of the present invention, with markings 909 to permit easy determination of the distance between collection mechanism 102 and balloon 901. Of course, markings 909 can also be used to appropriately index the location of collection mechanism 102 within body tract 900. In one embodiment, markings 909 can be provided with material which permits easy identification by imaging methods known in the art. For instance, the material used for markings can be fluorescent, luminescent, or any materials easily identified by imaging protocols available in the art such as by ultrasound, endoscopy, fluoroscopy etc. In various embodiments, elongate member 907 may serve any one or combination of purposes including, for example, adding longitudinal rigidity to collection mechanism 102, providing a conduit to which the distal portion of system 100 may be accessed, and/or providing a conduit for inflating and deflating a distal anchor balloon 901. Moreover, should it be desired, the embodiment illustrated in FIG. 9B or any of the FIGS. illustrated herein, can be configured with designs known in the art, so that movement of the collection mechanism 102 can be facilitated independent of the movement of the conveyance mechanism 104

It should be appreciated that although illustrated as being situated distal to collection mechanism 102, anchoring device (e.g., balloon 901) may alternatively be placed proximal to collection mechanism 102. In such an embodiment, balloon 901 can be expanded once collection mechanism 102 has been placed at the targeted site 902 to secure the position of collection mechanism 102 relative to the targeted site 902 and minimize or eliminate movement of collection mechanism 102 away from the targeted site 902 and out through opening 903.

Moreover, it should be appreciated that although disclosed as balloon 901, the anchoring device can generally embody any of the inflatable or expandable designs noted above in connection with collection mechanism 102, or any geometric designs, so long as such a design can expand to a diameter relatively larger than opening 903 at the esophageal junction to secure the anchoring device thereat. Furthermore, the anchoring device, as provided herein, can be employed in connection with any of the embodiments or designs disclosed or illustrated in the present application.

In operation, the system 100 of the present disclosure can be used for collection of materials within a body. Depending on the structure and configuration of the system 100, the collection mechanism (e.g., collection mechanism 102) can be transformable between at least one of the navigation state, a collection state, and a retrieval state. The different states can modify the collection mechanism 102 and/or other components of the system 100 to be used for a particular purpose. For example, the navigation state can modify the size and/or shape of the collection mechanism 102 for safely and comfortably navigate the collection mechanism 102 to a desired location within or out of the body. In another example, the collection state can modify the size and/or shape of the collection mechanism 102 for safely and comfortably using the collection mechanism 102 to collect desired sampled materials at a particular location. In another example, the retrieval state can modify the size and/or shape of the collection mechanism 102 for protecting any sampled materials while also providing a size and/or shape of the collection mechanism 102 for safely and comfortably removing the system 100 from a body. The retrieval state should also be reversable or modified such that any protected samples can be acquired once the system 100 has been removed from the body, for example, to be analyzed/tested.

The system 100 can be used for cell collection by first inserting the collection mechanism 102 within a body 108 while in the navigation state. The collection mechanism 102 can be navigated within the body 108 to a point of interest that includes an area to be sampled. After the collection mechanism 102 has been navigated to the target site for cell collection it can be activated by an activation mechanism 106. In some embodiments, the activation mechanism 106 can be used to initiate transformation of the collection mechanism 102 between navigation state to the collection state. Once in the collection state, the collection mechanism 102 is at the cell collection site, the collection mechanism 102 can be provided in contact with the body 108 to gather cells on at least one surface of the collection mechanism 102. In some embodiments, collection can be assisted by moving the collection mechanism 102 in various manners. For example, the collection mechanism 102 can be translated (i.e., moved back and forth) vertically or horizontally, /or rotated, or a combination thereof to best collect cells 110. Moreover, to optimize the collection of cells, the collection mechanism 102 can be made to translate vertically (i.e., axially) or horizontally along the vessel walls, such as body 108, depending on the location and orientation of the targeted site, from less than approximately 1 cm to approximately 5 cm about the targeted site. Additionally, or alternatively, the collection mechanism can be vibrated, for example, through the assistance of the activation mechanism 106.

It should be appreciated that the movement of the collection mechanism 102 can be achieved by movement of the conveyance mechanism 104 and collection mechanism 102 as an assembly. In another embodiment, the collection mechanism 102 can be moved individually and independently of the conveyance mechanism 104 by way of, for example, the activation mechanism 106. For example, activation mechanism 106 can be a wire or elongated member extending along the conveyance mechanism 104, and can be coupled at its distal end to collection mechanism 102. In that way, movement of the collection mechanism 102 can be controlled by the activation mechanism 106 independent of the conveyance mechanism 104.

Once collection has been achieved, the collection mechanism 102 can be transformed from a collection state to a retrieval state. The transformation can be initiated by activating the activation mechanism to transform of the collection mechanism 102 between the collection state and the retrieval state. When the collection mechanism 102 is in the retrieval state, any collected material can be positioned within the collection mechanism 102 such that it protects the collected cells. Thereafter, the collection mechanism 102 can be removed for retrieval and analysis of the collected materials (e.g., cells 110).

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A system for cell collection, the system comprising:
a collection mechanism being transformable between a collection state having an expanded diameter and a retrieval state where the diameter is less than that of the collection state;
a conveyance mechanism having a distal end to which the collection mechanism is attached, and designed to place the collection mechanism at a site of interest for cell collection; and
an activation mechanism to transform the collection mechanism between the collection state and the retrieval state, and to permit the collection mechanism to move independent of the conveyance mechanism.

2. The system of claim 1, wherein the activation mechanism is designed to control transformation of the cell collection mechanism between at least two of the collection state, the retrieval state and a navigation state.

3. The system of claim 1, wherein the cell collection mechanism includes:
an expandable material having a cell collection surface; and
a plurality of rings spaced along the expandable material and situated circumferentially around the expandable material,
wherein the expandable material is designed to expand from a first circumference less than a circumference of the plurality of rings to a second circumference greater than the circumference of the plurality of rings.

4. The system of claim 1, wherein the cell collection mechanism is a tubular construction with a plurality of struts to allow the cell collection mechanism to bow.

5. The system of claim 4, wherein the cell collection mechanism is designed to bow upon application of a axial force applied linearly along a central axis.

6. The system of claim 4, wherein the cell collection mechanism is designed to bow upon application of an internal force applied radial outward.

7. The system of claim 4, wherein the tubular construction is designed to bow upon application of heat energy.

8. The system of claim 1, wherein the cell collection mechanism is designed to expand upon application of heat energy.

9. The system of claim 1, wherein the cell collection mechanism is constructed with a plurality of creases and bends that are expandable into a shape for cell collection in the collection state and retracted for the retrieval state.

10. The system of claim 1, wherein the cell collection mechanism is a rigid tubular construction with a plurality slots to allow an internal expandable material to push through and expand out when in the collection state.

11. The system of claim 10, wherein the internal expandable material retracts into the plurality slots and within the rigid tubular construction when in the retrieval state.

12. The system of claim 1, wherein the cell collection mechanism is constructed from a plurality of tines that fan out from a central axis when in the collection state.

13. The system of claim 12, wherein the fan out of the plurality of tines is controlled by at least one of an expandable material, a slidable sheath, heat energy, or a combination thereof

14. The system of claim 12, wherein distal ends of the plurality of tines can retract to a smaller diameter than a remainder of the plurality of tines in the retrieval state.

15. The system of claim 1, wherein the cell collection mechanism includes a mandrel rod, and a plurality of arms for sampling.

16. The system of claim 1, further includes an imaging device proximate to the collection mechanism to assist in the localization and positioning of the collection mechanism at a targeted site.

17. The system of claim 1, further including an anchoring device adjacent to the collection mechanism to minimize movement of the collection mechanism.

18. The system of claim 17, wherein the anchoring device includes an expandable body.

19. A cell collection device, the device comprising:
a cell collection mechanism having a proximal end and a distal end, the cell collection mechanism being transformable between at least one of a navigation state, a collection state, and a retrieval state; and
a conveyance mechanism having a proximal end and a distal end, the distal end of the conveyance mechanism coupled to the proximal end of the cell collection mechanism.

20. A method for cell collection, the method comprising:
inserting a cell collection mechanism within a body in a navigation state, the cell collection mechanism being transformable between at least one of a navigation state, a collection state, and a retrieval state;
navigating the cell collection mechanism to a target site for cell collection;
activating an activation mechanism to transform of the cell collection mechanism between navigation state to the collection state;
moving the cell collection mechanism at the cell collection site to gather cells on at least one surface of the cell collection mechanism;
activating the activation mechanism to transform of the cell collection mechanism between the collection state and the retrieval state to protect the collected cells; and
removing the cell collection mechanism.

21. The method of claim 20, wherein, while the cell collection mechanism is in the retrieval state, the cell collection mechanism provides protection of the collected cells from dilution and contamination during retrieval.

22. A method for cell collection, the method comprising:
inserting, into an esophagus, an expandable cell collection mechanism coupled to a conveyance mechanism;
expanding the collection mechanism at a targeted site to permit contact between the collection mechanism and surrounding esophageal walls;
moving the collection mechanism while maintaining contact with the esophageal walls to collect cells at the targeted site; and
collapsing the collection mechanism.

23. The method of claim 22, wherein in the step of inserting, the collection mechanism is a balloon.

24. The method of claim 22, wherein in the step of inserting, the collection mechanism includes a metallic material.

25. The method of claim 22, wherein the step of moving includes translating the collection mechanism, rotating the collection mechanism, vibrating the collection mechanism, or a combination thereof

26. The method of claim 25, wherein the step of moving further includes translating the collection mechanism along the esophageal walls for at least 1 centimeter (cm).

27. The method of claim 22, wherein the step of moving further includes translating the collection mechanism along the esophageal walls for between at least about 2 cm to about 5 cm.

28. The method of claim 22, wherein the step of moving includes moving the conveyance mechanism to move the collection mechanism.

29. The method of claim 22, wherein the step of moving includes moving the collection mechanism independent of the conveyance mechanism.
